(19) United States
(12) Patent Application Publication
(10) Pub. No.: US 2024/0139546 A1
(43) Pub. Date: May 2, 2024

(54) NEUTRON CAPTURE THERAPY SYSTEM

(71) Applicant: NEUBORON THERAPY SYSTEM LTD., Xiamen (CN)

(72) Inventors: Di-yun SHU, Xiamen (CN); Qiu-Ping GONG, Xiamen (CN)

(21) Appl. No.: 18/399,913

(22) Filed: Dec. 29, 2023

Related U.S. Application Data
(63) Continuation of application No. PCT/CN2022/105433, filed on Jul. 13, 2022.

(30) Foreign Application Priority Data
Jul. 16, 2021 (CN) 202110807235.1

(51) Int. Cl. A61N 5/10

(52) U.S. Cl. CPC A61N 5/1065; A61N 5/1077; A61N 2005/109; A61N 2005/1094; A61N 2005/1095

(57) ABSTRACT

A neutron capture therapy system, which can reduce secondary radiation and radiation damage caused by the action of recoil neutrons generated in the process of generating neutron beams by a beam transmission portion and a neutron beam generation portion. The neutron capture therapy system comprises an accelerator, the beam transmission portion, and the neutron beam generation portion; the accelerator accelerates charged particles to generate charged particle beams; the beam transmission portion transmits the charged particle beams generated by the accelerator to the neutron beam generation portion; the neutron beam generation portion generates neutron beams for treatment; and the neutron capture therapy system comprises a beam transmission shielding component for the beam transmission portion.

### CROSS-REFERENCE TO RELATED PATENT APPLICATION

This application is a continuation application of International Application No. PCT/CN2022/105433, filed on Jul. 13, 2022, which claims priority to Chinese Patent Application No. 202110807235.1, filed on Jul. 16, 2021, the disclosures of which are hereby incorporated by reference.

### TECHNICAL FIELD

The present disclosure relates to a radiation irradiation system, in particular to a neutron capture therapy system.

### BACKGROUND

With the development of atomic science, radiation therapy, such as cobalt-60 radiation therapy, linear accelerator therapy and electron beam radiation therapy, has become one of the main means of cancer therapy. However, the traditional photon or electron therapy is limited by physical conditions of the radiation itself, which also damages a large amount of normal tissues in the beam path while killing tumor cells. In addition, due to different sensitivity of tumor cells to radiation, the traditional radiation therapy is often ineffective for therapy of malignant tumors with relatively high radiation resistance (e.g., glioblastoma multiforma and melanoma).

In order to reduce radiation damage to normal tissues surrounding tumors, the concept of target therapy in chemotherapy is applied to radiation therapy; for tumor cells with high radiation resistance, radiation sources with high relative biological effectiveness (RBE), such as proton therapy, heavy particle therapy and neutron capture therapy, are also actively developed at present. The neutron capture therapy combines the above two concepts, such as boron neutron capture therapy, which provides a better selection of cancer therapy than conventional radiation therapy by means of specific aggregation of a boron-containing drug in tumor cells, and in conjunction with precise neutron beam regulation.

Various radiations are generated during the radiation therapy, for example, neutrons and photons with energies ranging from low to high are generated during the boron neutron capture therapy, and these radiations may cause different degrees of damage to normal human tissues. Therefore, in the field of radiation therapy, how to reduce radiation pollution to an external environment, medical personnel or normal tissues of a patient while achieving effective treatment is an extremely important task.

Therefore, it is required to propose a new technical solution to solve the described problems.

### SUMMARY

In order to solve the foregoing technical problems, one aspect of the present disclosure provides a neutron capture therapy system, including an accelerator, a beam transmission portion, and a neutron beam generation portion, wherein the accelerator is configured for accelerating charged particles to generate a charged particle beam, the beam transmission portion transmits the charged particle beam generated by the accelerator to the neutron beam generation portion, the neutron beam generation portion generates a neutron beam for therapy, the neutron capture therapy system includes a beam transmission shielding assembly for the beam transmission portion. The beam transmission shielding assembly may reduce the secondary radiation and radiation damage caused by the recoil neutrons generated in the process of generating the neutron beam by the beam transmission portion and the neutron beam generation portion.

Preferably, the material of the beam transmission shielding assembly is boron-containing PE material.

Preferably, the beam transmission shielding assembly is at least partially movable or detachable.

Preferably, the beam transmission portion includes a transmission tube configured for accelerating or transmitting the charged particle beam, and the beam transmission shielding assembly includes a first shielding member surrounding the transmission tube. The first shielding member is an annular shielding sleeve surrounding the transmission tube.

Preferably, the beam transmission portion includes a transmission tube configured for accelerating or transmitting the charged particle beam and a beam adjusting portion arranged on the transmission tube and configured for adjusting the charged particle beam, the beam transmission shielding assembly includes a second shielding member arranged on the transmission tube, the second shielding member is located downstream of the beam adjusting portion in a transmission direction of the charged particle beam. The second shielding member is a shielding plate with a plate surface perpendicular to the transmission direction of the charged particle beam.

Furthermore, the beam transmission shielding assembly includes third shielding members arranged at two sides of the transmission tube in the transmission direction of the charged particle beam, the third shielding members include shielding plates arranged at two sides of the transmission tube in a direction parallel to the transmission direction of the charged particle beam. Furthermore, the second shielding member and the third shielding members are connected together and at least partially surround the beam adjusting portion and the transmission tube, so as to get a better shielding effect.

Furthermore, the beam adjusting portion includes an X-Y magnet configured for adjusting axes of the charged particle beam, or a quadrupole magnet configured for suppressing divergence of the charged particle beam, or a four-way septum magnet configured for shaping the charged particle beam, or a charged particle beam scanning magnet. The second shielding member includes at least one shielding plate located downstream of the magnet in the transmission direction of the charged particle beam.

Preferably, the neutron capture therapy system includes an irradiation chamber and a charged particle beam generation chamber, wherein a subject to be irradiated is performed therapy by means of irradiation of the neutron beam in the irradiation chamber, the charged particle beam generation chamber houses the accelerator and at least part of the beam transmission portion, and the beam transmission shielding assembly is arranged in the charged particle beam generation chamber.

Furthermore, the beam transmission portion includes a first transmission portion connected to the accelerator; a beam direction switcher switching a travel direction of the charged particle beam; and a second transmission portion transmitting the charged particle beam from the beam direction switcher to the neutron beam generation portion. The charged particle beam generation chamber includes an accelerator chamber and a beam transmission chamber, the first transmission portion extends from the accelerator chamber to the beam transmission chamber, the second transmission portion extends from the beam transmission chamber to the neutron beam generation portion, the neutron beam generation portion is at least partially arranged in a separation wall between the beam transmission chamber and the irradiation chamber, and the beam transmission shielding assembly is arranged in the beam transmission chamber.

Furthermore, the beam transmission shielding assembly includes a shielding casing surrounding the beam direction switcher.

Furthermore, the beam transmission portion includes a beam collector, the beam direction switcher selectively transmits the charged particle beam to the neutron beam generation portion or the beam collector. The beam transmission shielding assembly includes a first shielding plate arranged at an end of the beam collector, or a shielding cover arranged at a top of the beam collector, or a shielding sleeve surrounding the beam collector. Furthermore, each of the first transmission portion and the second transmission portion includes a transmission tube configured for accelerating or transmitting the charged particle beam and a beam adjusting portion arranged on the transmission tube and configured for adjusting the charged particle beam, the beam transmission shielding assembly includes second shielding plates respectively arranged on the transmission tubes of the first transmission portion and the second transmission portion and each having a plate surface perpendicular to a transmission direction of the charged particle beam, each of the second shielding plates is located downstream of the corresponding beam adjusting portion, and the beam transmission shielding assembly further includes third shielding plates arranged at two sides of the first transmission portion and the second transmission portion in the transmission direction of the charged particle beam, at least part of the second shielding plates, the first shielding plate and the third shielding plates are connected together and form a closed space together with the separation wall between the accelerator chamber and the beam transmission chamber, so as to shield the entire beam transmission portion in the closed space.

Preferably, the beam transmission portion includes a transmission auxiliary device, the transmission auxiliary device includes, but is not limited to, a vacuum pump, a cooling medium control mechanism, and an argon control mechanism. The beam transmission shielding assembly includes a forth shielding member at least partially surrounding or covering the transmission auxiliary device.

Preferably, the beam transmission shielding assembly further includes a portable shielding plate.

In the neutron capture therapy system of the present disclosure, the beam transmission shielding assembly is provided, so as to reduce the secondary radiation and radiation damage caused by the recoil neutrons generated in the process of generating the neutron beam by the beam transmission portion and the neutron beam generation portion.

### BRIEF DESCRIPTION OF THE DRAWINGS

FIG. 1 is a schematic structural view of a neutron capture therapy system according to an embodiment of the present disclosure;

FIG. 2 is a schematic layout view of a neutron capture therapy system in an embodiment of the present disclosure; and

FIG. 3 is a schematic view of an embodiment of a transmission tube shielding member for a neutron capture therapy system in an embodiment of the present disclosure.

### DETAILED DESCRIPTION OF THE EMBODIMENTS

Embodiments of the present disclosure is further described in detail below with reference to the accompanying drawings, so that those skilled in the art may implement the present disclosure according to the text of the specification.

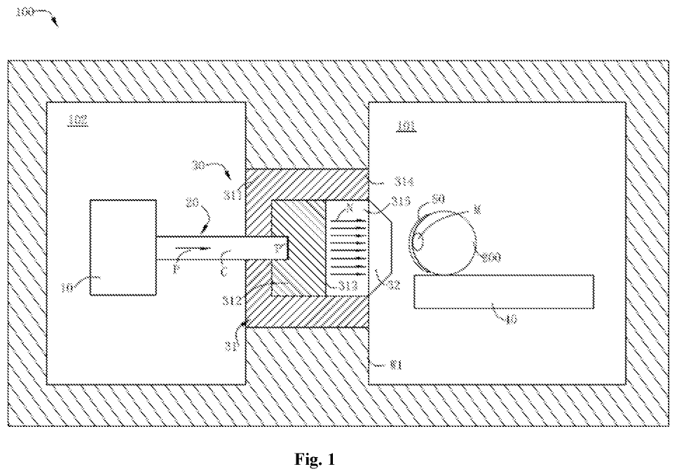

Referring to FIG. 1, a neutron capture therapy system in this embodiment is preferably a boron neutron capture therapy system 100. The boron neutron capture therapy system 100 is a device for performing cancer therapy by means of a boron neutron capture therapy. The boron neutron capture therapy carries out the cancer therapy by irradiating a neutron beam N to a patient 200 injected with boron (B-10). Upon taking or injecting the boron-containing (B-10) drug into the patient 200, the boron-containing drug selectively accumulates in tumor cells M. Based on a high capture cross section property for thermal neutrons of the boron-containing (B-10) drug, two heavy charged particles of ⁴He and ⁷Li are generated by a neutron capture and nuclear fission reaction of ¹⁰B(n, α)⁷Li. The average energy of the two charged particles of ⁴He and ⁷Li is about 2.33 MeV, and the two charged particles have high Linear Energy Transfer (LET) and short distance properties, wherein the Linear Energy Transfer and distance of the alpha particle are 150 keV/μm and 8 μm, respectively, while that of the heavy particle of ⁷Li are 175 keV/μm and 5 μm, respectively, the total distance of the two heavy particles is approximately equivalent to a cell size, and therefore the radiation damage to organisms is limited to the cell level, which can achieve the purpose of locally killing tumor cells without causing too much damage to normal tissues.

The boron neutron capture therapy system 100 includes an accelerator 10, a beam transmission portion 20, a neutron beam generation portion 30, and a treatment station 40. The accelerator 10 is configured for accelerating the charged particles (such as protons, deuterons) to generate a charged particle beam P such as a proton beam; the beam transmission portion 20 transmits the charged particle beam P generated by the accelerator to the neutron beam generation portion 30; the neutron beam generation portion 30 generates a neutron beam N for therapy and irradiates the patient 200 on the treatment station 40.

The neutron beam generation portion 30 includes a target T, a beam shaping portion 31, and a collimator 32. The charged particle beam P generated by the accelerator 10 irradiates the target T through the beam transmission portion 20 and interacts with the target T to generate neutrons. The generated neutrons form the neutron beam N for therapy through the beam shaping portion 31 and the collimator 32 in sequence, and irradiate the patient 200 on the treatment station 20. The target T is preferably a metal target. A suitable nuclear reaction is selected according to the required neutron yield and energy, energy and current of the available accelerated charged particles, and physical and chemical properties of the metal target, and the nuclear reactions commonly discussed are ⁷Li(p, n)⁷Be and ⁹Be(p, n)⁹B, which are both endothermic reactions. The energy threshold values of the two nuclear reactions are 1.881 MeV and 2.055 MeV, respectively, and since the ideal neutron source for the boron neutron capture therapy is epithermal neutrons of keV energy level, and theoretically, if the Li target is bombarded with protons whose energy is only slightly higher than the energy threshold value, it is possible to generate neutrons of relatively low energy, which can be used in the clinic without too much slow speed treatment, however, the proton action cross sections of the two targets of lithium metal (Li) and beryllium metal (Be) with the threshold energy are not high, therefore protons of higher energy are usually chosen to initiate the nuclear reaction in order to generate a sufficiently large flux of neutrons. An ideal target should have properties such as a high neutron yield, a distribution of energy of the generated neutron close to the energy region of the epithermal neutron (which will be described in detail below), no generation of too many long-distance radiation, low cost, safety, easy operation and high temperature resistance, but actually a nuclear reaction meeting all requirements cannot be found. As well known to a person skilled in the art, the target T may also be made of metal material other than Li and Be, such as Ta or W and an alloy thereof. The accelerator 10 may be a linear accelerator, a cyclotron, a synchrotron, a synchrocyclotron.

The beam shaping portion 31 may adjust the quality of the neutron beam N generated by the interaction of the charged particle beam P and the target T. The collimator 32 is configured for converging the neutron beam N, so that the neutron beam N has a higher targeting ability in the therapy process. The beam shaping portion 31 further includes a reflecting portion 311, a retarding portion 312, a thermal neutron absorbing portion 313, a radiation shielding portion 314, and a beam outlet 315. Neutrons generated by the interaction of the charged particle beam P and the target T have a wide energy spectrum, wherein in addition to the epithermal neutron meeting the therapy requirement, the proportion of other types of neutrons and photon needs to be minimized to avoid damage to the operator or the patient, therefore, the neutrons bombarded out from the target T need to pass through the retarding portion 312 to adjust the fast neutron energy (more than 40 keV) therein to the epithermal neutron energy region (0.5 eV-40 keV) and minimize thermal neutrons (less than 0.5 eV), the retarding portion 312 is made of a material having a larger action cross section with the fast neutron and a smaller action cross section with epithermal neutron, the retarding portion 312 is made of at least one of D₂O, AlF₃, Fluental™, CaF₂, Li₂CO₃, MgF₂ and Al₂O₃. The reflecting portion 311 surrounds the retarding portion 312, reflects neutrons passing through the retarding portion 312 and diffusing around back into the neutron beam N so as to improve the utilization rate of neutrons, and is made of a material having strong neutron-reflecting capacity. In the embodiment, the reflecting portion 311 is made of at least one of Pb or Ni. The thermal neutron absorbing portion 313 is arranged behind the retarding portion 312, and is made of a material having a large action cross section with the thermal neutron. In the embodiment, the thermal neutron absorbing portion 313 is made of Li-6, the thermal neutron absorbing portion 313 is configured for absorbing the thermal neutrons passing through the retarding portion 312, so as to reduce the proportion of the thermal neutrons in the neutron beam N, thus avoiding excessive doses from being applied to superficial normal tissues during the therapy. It's understandable that the thermal neutron absorbing portion may be integrated with the retarding portion, the material of the retarding portion contains Li-6. The radiation shielding portion 314 is configured for shielding neutrons and photons leaked from parts other than the beam outlet 315. The material of the radiation shielding portion 314 includes at least one of photon shielding material and neutron shielding material. In the embodiment, the material of the radiation shielding portion 314 includes photon shielding material plumbum (Pb) and neutron shielding material polyethylene (PE). It's understandable that the beam shaping portion 31 may have other configurations, as long as the epithermal neutron beam required for therapy may be obtained. The collimator 32 is arranged behind the beam outlet 1315, the epithermal neutron beam coming out from the collimator 32 irradiates the patient 200, pass through the superficial normal tissues, and then is retarded thermal neutrons to reach the tumor cells M. It's understandable that the collimator 32 may be cancelled or replaced by other structures, in this case, the neutron beam comes out from the beam outlet 315 and directly irradiates the patient 200. In the embodiment, a radiation shielding device 50 is further arranged between the patient 200 and the beam outlet 315, for shielding the radiation of the beam coming out from the beam outlet 315 to the normal tissues of the patient. It's understandable that the radiation shielding device 50 cannot be provided.

The target T is arranged between the beam transmission portion 20 and the beam shaping portion 31, and the beam transmission portion 20 has a transmission tube C for accelerating or transmitting the charged particle beam P. In the embodiment, the transmission tube C extends into the beam shaping portion 31 in the direction of the charged particle beam P, and sequentially passes through the reflecting portion 311 and the retarding portion 312, the target T is arranged in the retarding portion 312 and located at the end of the transmission tube C, so as to obtain a neutron beam with a better quality. Alternatively, the target may be arranged in other manners, for example, the target may be movable relative to the accelerator or the beam shaping portion, so as to facilitate replacement of the target or enable the charged particle beam to uniformly interact with the target.

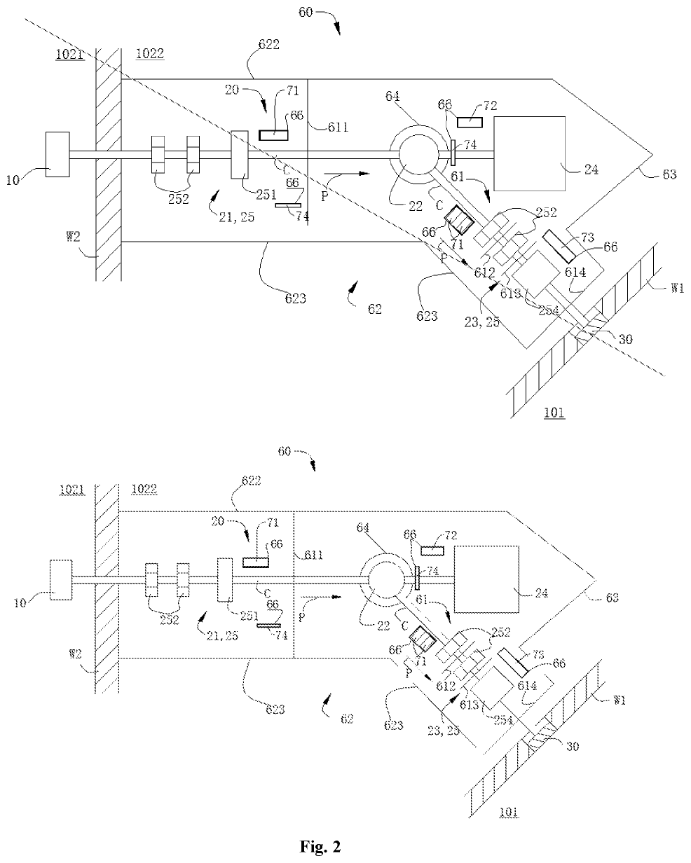

The boron neutron capture therapy system 100 is housed entirely within a concrete-constructed building. The boron neutron capture therapy system 100 includes an irradiation chamber 101 and a charged particle beam generation chamber 102. The patient 200 on the treatment station 20 is performed therapy by means of irradiation of the neutron beam N in the irradiation chamber 101, the charged particle beam generation chamber 102 houses the accelerator 10 and at least part of the beam transmission portion 20. In conjunction with FIG. 2, the beam transmission portion 20 includes a first transmission portion 21 connected to the accelerator 10; a beam direction switcher 22 switching a travel direction of the charged particle beam P; and a second transmission portion 23 transmitting the charged particle beam P from the beam direction switcher 22 to the neutron beam generation portion 30. The generated neutron beam N irradiates the patient 200 in the irradiation chamber 101. The beam transmission portion 20 may further include a beam collector 24 configured for, such as, collecting the beam when it is not be required and confirming whether the charged particle beam P is outputted before performing the therapy. The beam direction switcher 22 enable the charged particle beam P to be deviated from a normal track thereof and leaded to the beam collector 24 and the beam direction switcher 22 can selectively transmit the charged particle beam P to the neutron beam generation portion 30 or the beam collector 24.

The charged particle beam generation chamber 102 may include an accelerator chamber 1021 and a beam transmission chamber 1022, the first transmission portion 21 extends from the accelerator chamber 1021 to the beam transmission chamber 1022, the second transmission portion 23 extends from the beam transmission chamber 1022 to the neutron beam generation portion 30, and the neutron beam generation portion 30 is at least partially arranged in a separation wall W1 between the beam transmission chamber 1022 and the irradiation chamber 101. The beam collector 24 is arranged in the beam transmission chamber 1022. It's understandable that the beam collector 24 may be arranged in other positions, such as in a concrete wall.

The beam direction switcher 22 may include a deflection electromagnet configured for deflecting the travel direction of the charged particle beam P and a switching electromagnet configured for controlling the transmission direction of the charged particle beam P. Each of the first transmission portion 21 and the second transmission portion 23 includes the transmission tube C, and may include a beam adjusting portion 25 arranged on the transmission tube C and configured for adjusting the charged particle beam P. The beam adjusting portion 25 includes an X-Y magnet 251 configured for adjusting the axes of the charged particle beam P, a quadrupole magnet 252 configured for suppressing the divergence of the charged particle beam P, a four-way septum magnet 253 (not shown) configured for shaping the charged particle beam P, and the like. The beam adjusting portion 25 of the second transmission portion 23 may further include a charged particle beam scanning magnet 254 configured for scanning the charged particle beam P as required to control the irradiation of the charged particle beam P with respect to the target T, for example, to control the irradiation position of the charged particle beam P with respect to the target T.

Since a large amount of neutrons may be generated during a neutron capture therapy, especially near the target T that generates neutrons, it is required to avoid the neutron leakage as much as possible. In one embodiment, the concrete forming at least part of the space (e.g., the beam transmission chamber 1022 and the irradiation chamber 101) is concrete added with neutron shielding material, such as boron-containing barite concrete, to form a neutron shielding space. In another embodiment, surfaces of concrete inside the chamber (such as the beam transmission chamber 1022, a ceiling plate, a floor plate and a wall of the irradiation chamber 101,) are disposed with neutron shielding plates (not shown) such as a boron-containing PE plate to form a neutron shielding space. The emission directions of the neutrons generated by the interaction of the charged particle beam P and the target T are uniformly distributed in space. During the process of “shaping” neutrons by the beam shaping portion 31, a large amount of recoil neutrons, which are needed to be focused on in a radiation shielding design, are generated. The recoil neutrons are mainly concentrated around the beam transmission portion 20 of the beam transmission chamber 1022, and the material of the beam transmission portion 20 (such as the transmission tube C and the magnet arranged on the transmission tube C) is generally made of steel-containing materials which, when irradiated by the neutrons, generates a radioisotope with a long half-life, causing secondary radiation, a beam transmission shielding assembly 60 for the beam transmission portion 20 therefore is provided, so as to reduce the secondary radiation and radiation damage caused by the recoil neutrons generated in the process of generating the neutron beam by the beam transmission portion 20 and the neutron beam generation portion 30.

The beam transmission shielding assembly 60 is arranged in the beam transmission chamber 102 and includes a magnet shielding member 61 and a transmission tube shielding member 62. The magnet shielding member 61 is arranged on the transmission tube C, and is located downstream of the beam adjusting portion 25 in the transmission direction of the charged particle beam P. In one embodiment, the magnet shielding member 61 is a shielding plate with a plate surface perpendicular to the transmission direction of the charged particle beam P. Each magnet may be provided with one shielding plate, alternatively, the downstream of the magnet at the most downstream of each transmission tube C in the transmission direction of the charged particle beam P may be provided with a shielding plate, and the material of the magnet shielding member 61 is boron-containing PE material. In the embodiment, the beam adjusting portion 25 of the first transmission portion 21 includes one X-Y magnet 251 and two quadrupole magnets 252, and the beam adjusting portion 25 of the second transmission portion 23 includes two quadrupole magnets 252 and one charged particle beam scanning magnet 254, the magnet shielding member 61 includes a shielding plate 611 arranged on the transmission tube C between the X-Y magnet 251 of the first transmission portion 21 and the beam direction switcher 22, shielding plates 612, 613 arranged between each two of magnets of the second transmission portion 23, and a shielding plate 614 arranged between the charged particle beam scanning magnet 254 of the second transmission portion 23 and the neutron beam generation portion 30. Since the second transmission portion 23 is closer to the neutron beam generation portion 30, more magnet shielding members 61 are provided.

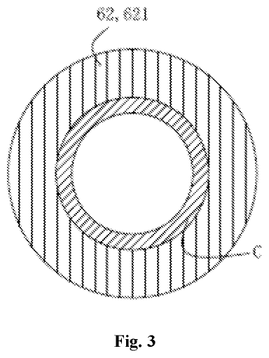

The transmission tube shielding member 62 is an annular shielding sleeve 621 (as shown in FIG. 3) surrounding the transmission tube C or includes shielding plates 622, 623 arranged at two sides of the transmission tube C in directions parallel to the transmission direction of the charged particle beam P. In order to reduce costs, the material of the transmission tube shielding member 62 may be PE material without boron. In the case that the transmission tube shielding member 62 includes the shielding plates 622, 623 arranged at two sides of the transmission tube C in directions parallel to the transmission direction of the charged particle beam P, the transmission tube shielding member 62 provides additional shielding for both the magnets and other components on the beam transmission portion 20. In one embodiment, at least part of the magnetic shielding member 61 and the transmission tube shielding member 62 may be connected together and at least partially surround the beam adjusting portion 25 and the transmission tube C, so as to achieve a better shielding effect.

The beam transmission shielding assembly 60 further includes a shielding plate 63 arranged at an end of the beam collector 24, the shielding plate 63 at least partially surrounds the other end oppose to the end of the beam collector 24 connected to the beam direction switcher 22, and the cross section of the shielding plate 63 may be is a shape of [-shaped or polygonal-shaped. In the case that the transmission tube shielding member 62 includes the shielding plates 622, 623 arranged at two sides of the transmission tube C in directions parallel to the transmission direction of the charged particle beam P, in one embodiment, at least part of the magnet shielding member 61, the transmission tube shielding member 62 and the shielding plate 63 are connected together and form a closed space together with the separation wall W2 between the accelerator chamber 1021 and the beam transmission chamber 1022, so that the entire beam transmission portion 20 in the closed space is shielded. In the embodiment, the shielding plate 622 arranged at one side of the transmission tube C, the shielding plate 63 arranged at the end of the beam collector chamber 24, the shielding plate 614 arranged between the charged particle beam scanning magnet 254 of the second transmission portion 23 and the neutron beam generation portion 30, the shielding plate 623 arranged at the other side of the transmission tube C, the separation wall W2 between the accelerator chamber 1021 and the beam transmission chamber 1022 are sequentially connected to form a closed space. The shielding plate 611 arranged between the X-Y magnet 251 of the first transmission portion 21 and the beam direction switcher 22 is connected to shielding plates 622 and 623 provided at two sides of the transmission tube C. Since the beam collector 24 contains a large mass of steel, in order to further reduce secondary radiation, a shielding cover (not shown) may be arranged at the top of the beam collector 24, alternatively, a shielding sleeve (not shown) may be arranged at the periphery of the beam collector 24 to surround the beam collector 24.

The beam direction switcher 22 may be surrounded by a shielding casing 64, so as to prevent the beam direction switcher 22 from interacting with the recoil neutrons to generate secondary radiation. The material of the shielding casing 64 may be boron-containing PE material.

The beam transmission shielding assembly 60 may further include a portable shielding plate 65 (not shown) which can be carried by an operator to move with the operator. The portable shielding plate 65 may further reduce radiation damage to the operator caused by secondary radiation generated by the beam transmission portion 20. It's understandable that the material of the portable shielding plate 65 is plumbum material or other photon shielding materials, and/or neutron shielding material.

The beam transmission portion 20 includes a transmission auxiliary device. The beam transmission shielding assembly 60 may further include an auxiliary shielding member 66 at least partially surrounding or covering the transmission auxiliary device. In the embodiment, the auxiliary shielding member 66 is a shielding box surrounding the transmission auxiliary device, and alternatively, a shielding casing or a shielding sleeve covering or surrounding the transmission auxiliary device. The auxiliary transmission device includes, but is not limited to, a first vacuum pump 71 and a second vacuum pump 72 configured for vacuuming, a control mechanism 73 (specifically, a water distribution tank) configured for controlling the on/off and flow rate of the cooling medium, and a control mechanism 74 (specifically, an argon tank) configured for controlling the on/off and flow rate of the argon gas. The material of the auxiliary shielding member 66 may be boron-containing PE material or other neutron shielding materials, and/or photon shielding material.

There may be one or more neutron beam generation portions 30, so as to generate one or more neutron beams N for therapy. Accordingly, the beam transmission portion 20 includes transmission portions transmitting the charged particle beams P to the neutron beam generation portions 30, while a same beam transmission shielding assembly 60 may be disposed for each of the transmission portions.

The boron neutron capture therapy system 100 may further include a preparation chamber, a control chamber, and other spaces configured for assisting the therapy.

It's understandable that, in this embodiment, the material of each of the concrete, the separation wall, the shielding plate, the shielding cover, the shielding sleeve, and the shielding casing may be replaced with other neutron shielding materials, and/or may be photon shielding materials; the shielding plate, the shielding cover, the shielding sleeve and the shielding casing may be mounted via an aluminum profile which, when irradiated by neutrons, generates a radioisotope with a short half-life. It's understandable that other materials can also be used. The shielding plate, the shielding cover, the shielding sleeve, and the shielding casing may be at least partially movable or detachable, which facilitates maintenance of the device.

Although the illustrative embodiments of the present disclosure have been described above to facilitate understanding of the present disclosure by those skilled in the art, however, it should be clear that the present disclosure is not limited to the specific embodiments, as long as such variations are within the spirit and scope of the disclosure as defined and determined by the appended claims, these variations are obvious and are within the scope of the present disclosure.

What is claimed is:

1. A neutron capture therapy system, comprising an accelerator, a beam transmission portion, and a neutron beam generation portion, wherein the accelerator is configured for accelerating charged particles to generate a charged particle beam, the beam transmission portion transmits the charged particle beam generated by the accelerator to the neutron beam generation portion, and the neutron beam generation portion generates a neutron beam for therapy, characterized in that: the neutron capture therapy system comprises a beam transmission shielding assembly for the beam transmission portion.

2. The neutron capture therapy system according to claim 1, wherein the beam transmission shielding assembly is at least partially movable or detachable.

3. The neutron capture therapy system according to claim 1, wherein the beam transmission portion comprises a transmission tube configured for accelerating or transmitting the charged particle beam, and the beam transmission shielding assembly comprises a first shielding member surrounding the transmission tube.

4. The neutron capture therapy system according to claim 1, wherein the beam transmission portion comprises a transmission tube configured for accelerating or transmitting the charged particle beam and a beam adjusting portion arranged on the transmission tube to adjust the charged particle beam, the beam transmission shielding assembly comprises a second shielding member arranged on the transmission tube, and the second shielding member is located downstream of the beam adjusting portion in a transmission direction of the charged particle beam.

5. The neutron capture therapy system according to claim 4, wherein the second shielding member is a shielding plate with a plate surface perpendicular to the transmission direction of the charged particle beam.

6. The neutron capture therapy system according to claim 4, wherein the beam transmission shielding assembly comprises third shielding members arranged at two sides of the transmission tube in the transmission direction of the charged particle beam, the second shielding member and the third shielding members are connected and at least partially surround the beam adjusting portion and the transmission tube.

7. The neutron capture therapy system according to claim 6, wherein the third shielding members comprise shielding plates arranged at two sides of the transmission tube in a direction parallel to the transmission direction of the charged particle beam.

8. The neutron capture therapy system according to claim 4, wherein the beam adjusting portion comprises an X-Y magnet configured for adjusting axes of the charged particle beam, or a quadrupole magnet configured for suppressing divergence of the charged particle beam, or a four-way septum magnet configured for shaping the charged particle beam P, or a charged particle beam scanning magnet; and the second shielding member comprises at least one shielding plate located downstream of the magnet in the transmission direction of the charged particle beam.

9. The neutron capture therapy system according to claim 6, wherein the material of the second shielding member is boron-containing PE material, and the material of the third shielding member is PE material.

10. The neutron capture therapy system according to claim 1, wherein the neutron capture therapy system comprises an irradiation chamber and a charged particle beam generation chamber, wherein a subject to be irradiated is performed therapy by means of irradiation of the neutron beam in the irradiation chamber, the charged particle beam generation chamber houses the accelerator and at least part of the beam transmission portion, and the beam transmission shielding assembly is arranged in the charged particle beam generation chamber.

11. The neutron capture therapy system according to claim 10, wherein the beam transmission portion comprises a first transmission portion connected to the accelerator; a beam direction switcher switching a travel direction of the charged particle beam; and a second transmission portion transmitting the charged particle beam from the beam direction switcher to the neutron beam generation portion; and wherein the charged particle beam generation chamber comprises an accelerator chamber and a beam transmission chamber, the first transmission portion extends from the accelerator chamber to the beam transmission chamber, the second transmission portion extends from the beam transmission chamber to the neutron beam generation portion, the neutron beam generation portion is at least partially arranged in a separation wall between the beam transmission chamber and the irradiation chamber, and the beam transmission shielding assembly is arranged in the beam transmission chamber.

12. The neutron capture therapy system according to claim 11, wherein the beam transmission shielding assembly comprises a shielding casing surrounding the beam direction switcher.

13. The neutron capture therapy system according to claim 12, wherein the material of the shielding casing is boron-containing PE material.

14. The neutron capture therapy system according to claim 11, wherein the beam transmission portion comprises a beam collector, the beam direction switcher selectively transmits the charged particle beam to the neutron beam generation portion or the beam collector; and wherein the beam transmission shielding assembly comprises a first shielding plate arranged at an end of the beam collector, or a shielding cover arranged at a top of the beam collector, or a shielding sleeve surrounding the beam collector.

15. The neutron capture therapy system according to claim 14, wherein each of the first transmission portion and the second transmission portion comprises a transmission tube configured for accelerating or transmitting the charged particle beam and a beam adjusting portion arranged on the transmission tube and configured for adjusting the charged particle beam; the beam transmission shielding assembly comprises second shielding plates respectively arranged on transmission tubes of the first transmission portion and the second transmission portion and each having a plate surface perpendicular to a transmission direction of the charged particle beam, each of the second shielding plates is located downstream of corresponding beam adjusting portion; and the beam transmission shielding assembly further comprises third shielding plates arranged at two sides of the first transmission portion and the second transmission portion in the transmission direction of the charged particle beam, and at least part of the second shielding plates, the first shielding plate and the third shielding plates are connected together and form a closed space together with the separation wall between the accelerator chamber and the beam transmission chamber.

16. The neutron capture therapy system according to claim 15, wherein the first shielding plate, the third shielding plates, the second shielding plate configured between the neutron beam generation portion and the beam adjusting portion arranged on the second transmission portion, the separation wall W2 between the accelerator chamber and the beam transmission chamber are sequentially connected to form a closed space.

17. The neutron capture therapy system according to claim 1, wherein the beam transmission portion further comprises a transmission auxiliary device; and the beam transmission shielding assembly comprises a forth shielding member at least partially surrounding or covering the transmission auxiliary device.

18. The neutron capture therapy system according to claim 17, wherein the material of the forth shielding member is boron-containing PE material.

19. The neutron capture therapy system according to claim 1, wherein the beam transmission shielding assembly further comprises a portable shielding plate.

20. The neutron capture therapy system according to claim 19, wherein the material of the portable shielding plate is plumbum material.
